(19) United States
(12) Patent Application Publication
(10) Pub. No.: US 2018/0161028 A1
(43) Pub. Date: Jun. 14, 2018

(54) CUTTING TOOL

(71) Applicant: DEPUY SYNTHES PRODUCTS, INC., Raynham, MA (US)

(72) Inventors: Simon M. Bosshard, Bern (CH); This AEBI, Grenchen (CH)

(21) Appl. No.: 15/374,801

(22) Filed: Dec. 9, 2016

(51) Int. Cl. A61B 17/04; A61B 17/88; A61B 17/3205; A61B 17/3201

(52) U.S. Cl. CPC A61B 17/0467; A61B 17/8863; A61B 17/0469; A61F 2002/0817; A61B 17/3201; A61B 2017/0409; A61B 2017/0464; A61B 17/32053

(57) ABSTRACT

The various embodiments relate to cutting tools including non-shearing cutting blades which allow in-line cutting of various materials. The cutting tools further include handles, pivot assemblies, and a capture space for removing cut materials. In use, the cutting blades are brought together, forming a capture space that holds materials cut by the cutting blades. The cut material does not fly off or become lost in the patient and can be removed within the capture space. Various embodiments of the cutting tools include cutting blades angled inward towards an inner surface. Various embodiments further relate to methods of using the cutting tools for removing crimped cable ends, suture knots or soft tissue from a patient.

### TECHNICAL FIELD

Various embodiments disclosed herein relate generally to cutting tools, particularly to a device for minimally invasively inline cutting of a crimped end of a surgical cable.

### BACKGROUND

Orthopedic surgical procedures often require bone to be anchored to another piece of bone or soft tissue. Attachment systems, including bone bolts and cables encircling pieces of bone, may require large access incisions. A cable that circles the bone additionally may require the removal of more soft tissue to gain access to the bone. Larger incisions can increase patient pain and require more recovery time or be impractical for some target sites. Therefore, small incision procedures increase patient comfort and decrease recovery time.

Anchoring systems allow a surgeon to hold bone and soft tissue together through a less invasive operation. Such systems are taught by Cole et al. (U.S. Pat. No. 6,544,267). In these systems, a cable is inserted through the bone and soft tissue to be connected. Tension is applied to the cable, bringing the bone and soft tissue together. The end of the cable is then crimped to hold the cable in place. Later the anchoring system may be removed from the patient. To remove the tensioning cable, the crimped end portion may be cut away from the cable, allowing removal of the entire cable.

In general, cutting tools used to cut cables inside a patient cut the cable ends from a side of the cable using shearing blades. Cutting the cable from the side requires a larger incision so that the cutting tool can be placed at an angle to cut a crimped cable end attached to the bone. The crimped end portion of the cable is cut off to facilitate removal of the cable previously attached in a patient. The crimped end portion of the cable will thus be cut off and must be removed from the body. As a minimally invasive procedure is beneficial over surgery requiring a large incision, a cutting tool that may be used in minimally sized incisions is desirable.

### SUMMARY OF THE INVENTION

In light of the present need for a minimally invasive cutting tool, a brief summary of various exemplary embodiments is presented. Some simplifications and omissions may be made in the following summary, which is intended to highlight and introduce some aspects of the various exemplary embodiments, but not to limit the scope of the invention. Detailed descriptions of a preferred exemplary embodiment adequate to allow those of ordinary skill in the art to make and use the inventive concepts will follow in later sections.

Various embodiments disclosed herein relate to a cutting tool that includes a first handle, a second handle, and a pivot assembly joining the first handle and the second handle. A first head part extends from the first handle and a second head part extends from the second handle, wherein the first head part includes a first blade and the second head part includes a second blade such that when the first head part and the second head part are brought together, the first blade and the second blade are aligned, creating a capture space.

Various embodiments disclosed herein relate to a cutting tool that includes a first handle extension extending from the first handle and a second handle extension extending from the second handle.

Various embodiments disclosed herein relate to a cutting tool that includes pivot assemblies joining the first handle with the first head part and the second handle with the second head part. A fourth pivot assembly joins the first head part and the second head part.

Various embodiments disclosed herein relate to a cutting tool that is configured to cut by clamping together the first and second cutting blades.

Various embodiments disclosed herein relate to a cutting tool that includes first and second cutting blades that are continuous. Various embodiments include first and second cutting blades that are substantially a rectangular shape. Various embodiments include first and second cutting blades that extend along all sides of the substantially rectangular shape. Various embodiments include first and second cutting blades that do not extend along all sides of a substantially rectangular shape.

Various embodiments include first and second cutting blades that are a shape other than rectangular. Various embodiments include first and second cutting blades that extend along all sides of a shape other than rectangular. Various embodiments include first and second cutting blades that do not extend along all sides of a shape other than rectangular. Various embodiments disclosed herein relate to a cutting tool that includes a first blade that is angled inward towards an inner surface of a first head part. Various embodiments include a first blade that is angled inward at an angle of about 40° to about 50°. Various embodiments include a first blade that is angled inward at an angle of about 44° to about 46°. Various embodiments include a second blade that is angled inward towards an inner surface of a second head part. Various embodiments include a second blade that is angled inward at an angle of about 40° to about 50°. Various embodiments include a second blade that is angled inward at an angle of about 44° to about 46°.

Various embodiments disclosed herein relate to a cutting tool that includes a capture space that is fully enclosed.

Various embodiments disclosed herein relate to a cutting tool that includes a capture space that is not fully enclosed.

Various embodiments disclosed herein relate to a cutting tool that includes first and second cutting blades that extend along three sides of a substantially rectangular shape.

Various embodiments disclosed herein relate to a cutting tool that includes first and second cutting blades that are convex or concave at a distal end.

Various embodiments disclosed herein relate to a cutting tool that includes first and second cutting blades that are not convex or concave at a distal end.

Various embodiments disclosed herein relate to a cutting tool that includes a first head part with a first inner surface such that the first blade and first inner surface create a first partial capture space. The second head part includes a second inner surface such that the second blade and second inner surface create a second partial capture space. The first partial capture space and the second capture space form the capture space.

Various embodiments disclosed herein relate to a cutting tool that includes a first partial capture space with a first depth and a second partial capture space with a second depth, wherein the first depth is equivalent to the second depth.

Various embodiments disclosed herein relate to a cutting tool that includes a first partial capture space with a first depth and a second partial capture space with a second depth, wherein the first depth is not equivalent to the second depth.

Various embodiments disclosed herein relate to a cutting tool having a first depth of about 0 mm. Various embodiments relate to a cutting tool having a second depth of about 0 mm.

Various embodiments disclosed herein relate to a method of preparing a cutting tool for reprocessing that does not include decomposing the cutting tool.

Various embodiments disclosed herein relate to a method of cutting a closure within a living body that includes providing a cutting tool having a pair of handles and first and second cutting blades. The pair of handles is actuated to bring together the first and second cutting blades to cut away the closure which is held between the first and second cutting blades for removal from the living body.

Various embodiments disclosed herein relate to a method of cutting a crimped cable end within a living body that includes placing the cutting tool to allow for in-line cutting of the crimped cable end. Various embodiments include placing the cutting tool to allow for non-in-line cutting of the crimped cable end.

Various embodiments disclosed herein relate to a method of cutting a closure within a living body that includes cutting a closure that is a crimped portion of a crimped cable end. Various embodiments include cutting a closure that is a knot portion of a suture or a rope.

Various embodiments disclosed herein relate to a method of removing soft tissue from a patient that includes providing a cutting tool having a pair of handles and first and second cutting blades. The pair of handles is actuated to bring together the first and second cutting blades to cut away the soft tissue which is held between the first and second cutting blades in a capture space for removal from the patient.

Various embodiments disclosed herein relate to a method of removing soft tissue from a patient that includes placing the cutting tool to allow for in-line cutting of the soft tissue. Various embodiments include placing the cutting tool to allow for non-in-line cutting of the soft tissue.

### BRIEF DESCRIPTION OF THE DRAWINGS

In order to better understand various exemplary embodiments, reference is made to the accompanying drawings, wherein:

FIG. 1 is a side view of an embodiment of an open, relaxed cutting tool.

FIG. 2 is a side view of the cutting tool when actuated.

FIG. 3 shows the first and second head parts of the cutting tool.

FIG. 4 shows a cross-section of the first and second head parts of the cutting tool.

FIG. 5 illustrates in-line cutting of a crimped cable end using the cutting tool.

### DETAILED DESCRIPTION

The description and drawings presented herein illustrate various principles. It will be appreciated that those skilled in the art will be able to devise various arrangements that, although not explicitly described or shown herein, embody these principles and are included within the scope of this disclosure. As used herein, the term, “or” refers to a non-exclusive or (i.e., and/or), unless otherwise indicted (e.g., “or else” or “or in the alternative”). Additionally, the various embodiments described herein are not necessarily mutually exclusive and may be combined to produce additional embodiments that incorporate the principles described herein.

The embodiments described herein provide for cutting tools used in minimally invasive procedures. The cutting tool for minimally invasive procedures can cut in-line, which does not require a large incision. The cutting tool blades of the various embodiments do not shear and will not break or separate on soft tissue that may be in the path of cutting blades. The clamping motion of the blades further allows improved cutting of flexible cables that do not cut well with shearing blades. The cutting tool thus may be used for removing a number of materials, including cable crimps, suture knots or biopsies.

The cutting tool shape captures the cut material in a capture space. The cut material may include the crimped end of a surgical cable and/or soft tissue material located between the blades. Alternatively, the cut material may include various closures and materials. The cut material may include a knot portion from a suture or rope. The captured material may be pulled out of the body within the cutting tool. Where the blades cut around more than one side of the head part, the cutting tool does not need to be as precisely placed at a cutting site. Various embodiments of the cutting tool include a blade on more than one side of the head part which allows for cutting that may be in-line with a cable or from the side of material to be cut. The various cut materials and location may require various cutting angles using the cutting tool.

Described herein are various embodiments of the cutting tool that include blades angled inward toward the capture area of the head part. The blade placement and form allows for grinding the blades without dismantling the cutting tool. Thus, the cutting tool can be prepared for reuse without decomposing the cutting tool.

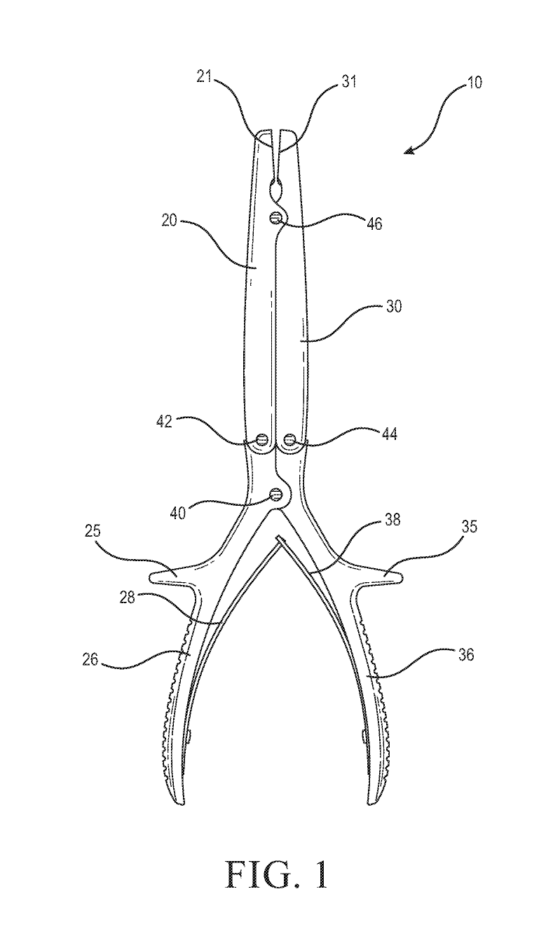

Referring now to the drawings, in which like numerals refer to like components or steps, there are disclosed broad aspects of various exemplary embodiments. FIG. 1 illustrates an embodiment of the cutting tool 10 in an open and relaxed position. The cutting tool 10 includes a pair of handles 26, 36 which optionally include extensions 25, 35 extending therefrom. Handle extensions 25, 35 provide grip support for a user of the cutting tool 10. More specifically, the handle extensions 25, 35 allow the user to apply a force on the cutting tool 10 towards the proximal end of the cutting tool 10 without the user's hand slipping. The application of this force by the user facilitates the cutting tool 10 cutting the crimped end of a cable at a desired position. The embodiment of FIG. 1 includes spring pieces 28, 38 located between the handles 26, 36. First handle 26 is connected to second handle 36 at first pivot assembly 40. In this embodiment, first handle 26 is further connected to the proximal end of first head part 20 at second pivot assembly 42 and second handle 36 is connected to the proximal end of second head part 30 at third pivot assembly 44. Accordingly, the first head part 20 extends from the first handle 26, and the second head part 30 extends from the second handle 36. Towards the distal end of head parts 20, 30, first head part 20 and second head part 30 are connected at fourth pivot assembly 46. Alternative embodiments may include various numbers of pivot assemblies. The pivot assemblies shown in FIG. 1 are arranged to provide a mechanical advantage in a smaller operating space as compared to, for example, a single pivot, such that the mechanical advantage leads to increased cutting force. Further, if a single pivot assembly is used between the first handle 26 and the second handle 36, then the first head part 20 and the second head part 30 may be integral to the first handle 26 and the second handle 36, respectively. Each of pivot assemblies 40, 42, 44, 46 includes an attachment. The attachment may include pins, screws, nuts and bolts, rivets, soldering, or combinations thereof. First and second cutting blades 21, 31 are part of the distal end of head parts 20, 30 and further include blade edges.

In use, first handle 26 and second handle 36 are actuated, with the first handle 26 and second handle 36 rotating about the first pivot assembly 40. This pivoting about the first pivot assembly 40 causes distal ends of the first handle 26 and the second handle 36 to separate, which causes the proximal ends of the first head part 20 and the second head part 30 to separate via the pivot assemblies 42, 44, and cause the first head part 20 and the second head part 30 to rotate about the fourth pivot assembly 46. The pivoting of the first head part 20 and the second head part 30 moves the first and second cutting blades 21, 31 toward each other, causing the first and second cutting blades 21, 31 to cut through any item or material between them.

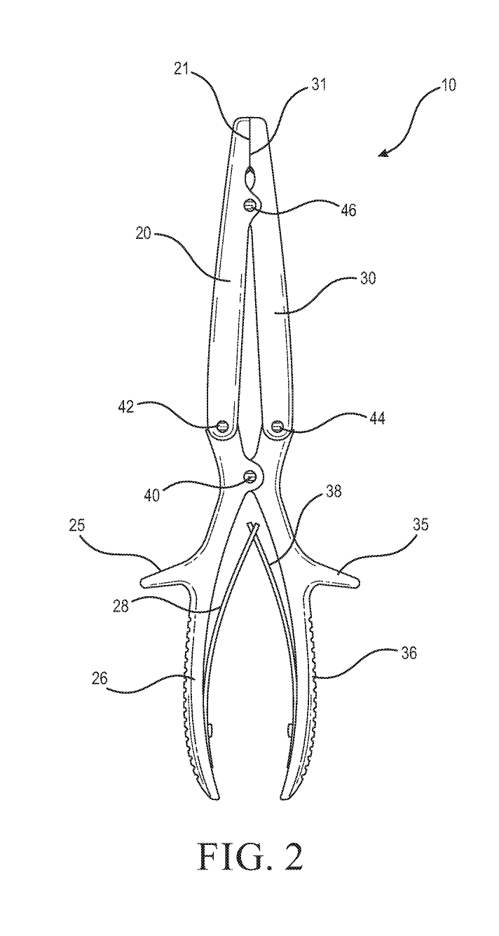

FIG. 2 illustrates an embodiment of the cutting tool 10 in an actuated position. The various elements labelled in FIG. 2 have the same name and description as above. The cutting tool 10 handles 26, 36 are actuated by being pulled towards each other by the user. The handles 26, 36 may be actuated with a single hand, allowing more flexibility to the user. The actuation engages the pivot assemblies 40, 42, 44, 46 which bring together the distal ends of the head parts 20, 30. The first and second cutting blades 21, 31 have complementary shapes and are brought together by the actuation of the cutting tool 10. Spring pieces 28, 38 return the cutting tool 10 to its relaxed position, shown in FIG. 1, when the user removes pressure from the handles 26, 36. While two separate connected spring pieces 28, 38 are shown, a single spring piece may also be used have a shape generally similar to the connected spring pieces 28, 38.

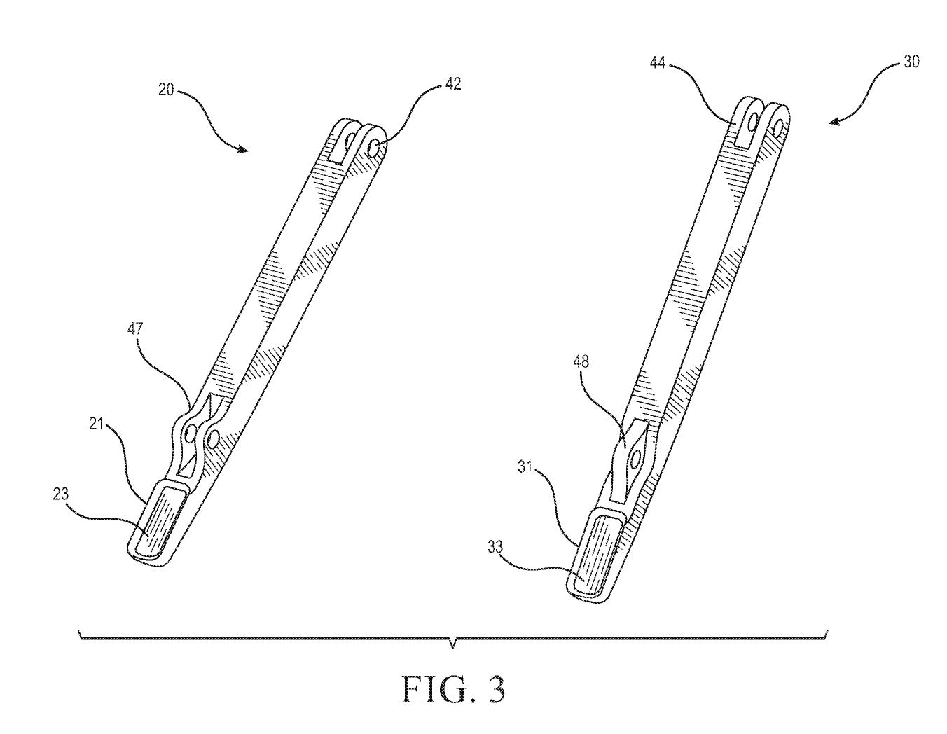

FIG. 3 illustrates an embodiment of the head parts 20, 30. The various elements labelled in FIG. 3 have the same name and description as above. The first head part 20 includes first connection point 47 which is joined with the second connection point 48 on the second head part 30 to form the fourth pivot assembly 46. First and second cutting blades 21, 31 are at the distal end of the head parts 20, 30. First cutting blade 21 and second cutting blade 31 are substantially rectangular and continuous as illustrated. Alternatively, the first cutting blade 21, second cutting blade 31 and blade edges may be a number of shapes and sizes. For example, nonexhaustively, the first cutting blade 21 and second cutting blade 31 may be circular or triangular. A nonexhaustive list of blade edge shapes along any axis includes straight, notched, or jagged blades. The distal end of the first and second cutting blades 21, 31 may be convex or concave, which allows for in-line or wide angle cutting. Alternatively, the distal end of the first and second cutting blades 21, 31 may be a shape other than convex or concave. The first cutting blade 21 and second cutting blade 31 may be continuous or non-continuous. For example, the first and second cutting blades 21, 31 may extend along only three sides of the rectangle, such that there is no blade on the side near the first connection point 47 and the second connection point 48. In such an arrangement, the structures around the first connection point 47 and the second connection point 48 help to create a capture space as discussed below.

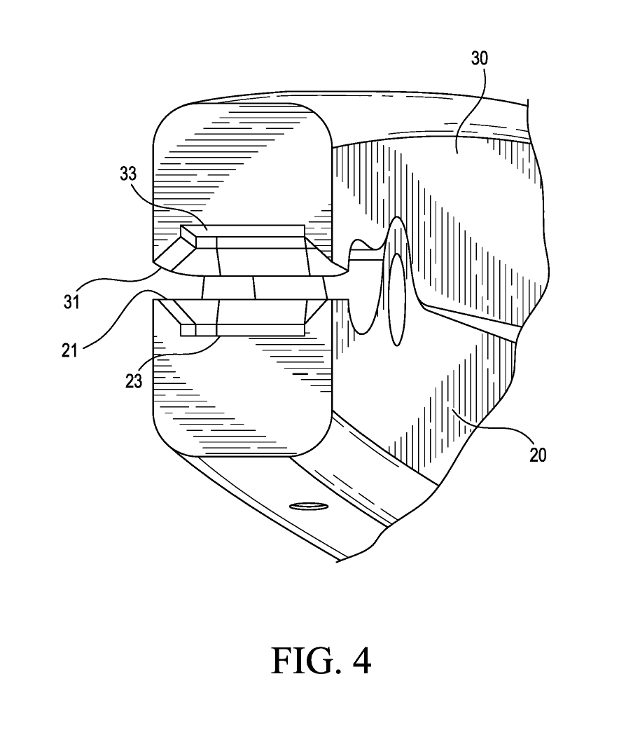

FIG. 4 illustrates a cross-section of an embodiment of the head parts 20, 30. The various elements labelled in FIG. 4 have the same name and description as above. In the illustrated embodiment, the first cutting blade 21 angles inward toward the first inner surface 23 and the second cutting blade 31 angles inward toward the second inner surface 33. The blade angles are preferably from about 40° to about 50° and more preferably from about 44° to about 46°. The blade configuration allows for reuse of the cutting tool 10 without decomposition of the cutting tool 10. The first and second cutting blades 21, 31 may be sharpened without decomposition of the cutting tool 10 due to the blade angles. First cutting blade 21 on the first head part 20 and second cutting blade 31 on the second head part 30 are complementary shapes such that when the head parts 20, 30 are brought together, the first and second cutting blades 21, 31 align. A first partial capture space is defined by the first cutting blade 21 and the first inner surface 23, and a second partial capture space is defined by the second cutting blade 31 and the second inner surface 33. A capture space is formed between the first and second cutting blades 21, 31 and the inner surfaces 23, 33 when the cutting tool 10 is actuated. The capture space includes the first partial capture space and the second partial capture space. The first partial capture space has a first depth and the second capture space has a second depth. The first depth is measured from the edge of the first cutting blade 21 to the first inner surface 23. The second depth is measured from the edge of the second cutting blade 31 to the second inner surface 33. The first depth and the second depth may be the same or different. In alternative embodiments, the first cutting blade 21 may be flat such that the first depth is 0 mm or the second cutting blade 31 may be flat such that the second depth is 0 mm. Material that is cut, including crimped cables, suture knots or soft tissue, is held in the capture space and may be removed from the body. The capture space provides the benefit of preventing the cut crimped portion of the cable from falling into the patient or flying off and injuring personnel in the operating room. It allows for the cutting and capture of tissue from the patient as needed, which then may be further examined and tested.

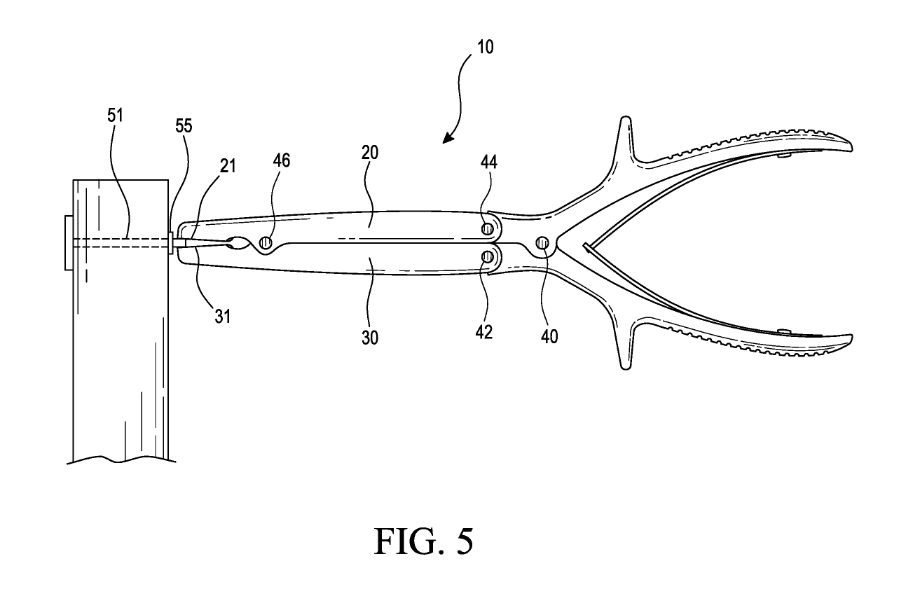

FIG. 5 illustrates in-line cutting of a crimped cable end 51 using the cutting tool 10. The various elements labelled in FIG. 5 have the same name and description as above. The illustrated embodiment of the cutting tool 10 includes first and second cutting blades 21, 31 at the distal end of the first head part 20 and the second head part 30 which allow for in-line cutting at the distal end of the cutting tool 10. The first and second cutting blades 21, 31 may be placed around a crimped portion 55 of the crimped cable end 51 to cut the crimped portion 55 away from the crimped cable 51. The crimped portion 55 may be cut without placing the cutting tool 10 at a wide angle from an axis along the crimped cable end 51. Alternatively, the cutting tool 10 may cut the crimped portion 55 at a wide angle from an axis along the crimped cable end 51. An alternative method may require cutting the crimped portion 55 from the side which may require a more invasive procedure and a larger incision. In-line cutting of the crimped cable end 51 may require smaller incisions that are beneficial for patient recovery.

Although the various exemplary embodiments have been described in detail with particular reference to certain exemplary aspects thereof, it should be understood that the invention is capable of other embodiments and its details are capable of modifications in various obvious respects. As is readily apparent to those skilled in the art, variations and modifications can be affected while remaining within the spirit and scope of the invention. Accordingly, the foregoing disclosure, description, and figures are for illustrative purposes only and do not in any way limit the invention, which is defined only by the claims.

What is claimed is:

1. A cutting tool, the cutting tool comprising:
a first handle and a second handle;
a pivot assembly joining the first handle and the second handle;
a first head part extended from the first handle and a second head part extended from the second handle, wherein the first head part includes a first cutting blade and the second head part includes a second cutting blade such that when the first head part and the second head part are brought together, the first cutting blade and the second cutting blade are aligned, creating a capture space.

2. The cutting tool of claim 1, further comprising a first handle extension extending from the first handle and a second handle extension extending from the second handle.

3. The cutting tool of claim 1, further comprising a second pivot assembly joining the first handle and the first head part, a third pivot assembly joining the second handle and the second head part, and a fourth pivot assembly joining the first head part and the second head part.

4. The cutting tool of claim 1, wherein the cutting tool is configured to cut by clamping together the first and second cutting blades.

5. The cutting tool of claim 1, wherein the first and second cutting blades are continuous.

6. The cutting tool of claim 1, wherein the first and second cutting blades are substantially a rectangular shape.

7. The cutting tool of claim 6, wherein the first and second cutting blades extends along all sides of the substantially rectangular shape.

8. The cutting tool of claim 1, wherein the first and second cutting blades are substantially a shape other than rectangular.

9. The cutting tool of claim 8, wherein the first and second cutting blades extend along all sides of the shape other than rectangular.

10. The cutting tool of claim 1, wherein the first cutting blade is angled inward towards an inner surface of the first head part

11. The cutting tool of claim 10, wherein the first cutting blade is angled inward at an angle of about 40° to about 50°.

12. The cutting tool of claim 11, wherein the first cutting blade is angled inward at an angle of about 44° to about 46°.

13. The cutting tool of claim 1, wherein the second cutting blade is angled inward towards an inner surface of the second head part.

14. The cutting tool of claim 13, wherein the second cutting blade is angled inward at an angle of about 40° to about 50°.

15. The cutting tool of claim 14, wherein the second cutting blade is angled inward at an angle of about 44° to about 46°.

16. The cutting tool of claim 1, wherein the capture space is fully enclosed.

17. The cutting tool of claim 1, wherein the capture space is not fully enclosed.

18. The cutting tool of claim 1, wherein the first and second cutting blades extend along three sides of a substantially rectangular shape.

19. The cutting tool of claim 1, wherein distal ends of the first and second cutting blades are convex or concave.

20. The cutting tool of claim 1, wherein distal ends of the first and second cutting blades are not convex or concave.

21. The cutting tool of claim 1, wherein
the first head part includes a first inner surface and the first cutting blade and first inner surface create a first partial capture space,
the second head part includes a second inner surface and the second cutting blade and second inner surface create a second partial capture space, and
the first partial capture space and the second partial capture space form the capture space.

22. The cutting tool of claim 21, wherein the first partial capture space has a first depth and the second partial capture space has a second depth, wherein the first depth is equivalent to the second depth.

23. The cutting tool of claim 21, wherein the first partial capture space has a first depth and the second partial capture space has a second depth, wherein the first depth is not equivalent to the second depth.

24. The cutting tool of claim 23,wherein the first depth is about 0 mm.

25. The cutting tool of claim 23, wherein the second depth is about 0 mm.

26. A method of preparing the cutting tool of claim 1 for reprocessing where the cutting tool is not decomposed.

27. A method of cutting a closure within a living body, the method comprising:
providing a cutting tool having a pair of handles and first and second cutting blades, the first and second cutting blades facing each other;
actuating the pair of handles to bring together the first and second cutting blades to cut away the closure, wherein
the closure is held between the first and second cutting blades in a capture space for removal from the living body.

28. The method of claim 27, wherein the cutting tool is placed to allow in-line cutting of the closure.

29. The method of claim 27, wherein the cutting tool is placed to allow non-in-line cutting of the closure.

30. The method of claim 27, wherein the closure is a crimped portion of a crimped cable end.

31. The method of claim 27, wherein the closure is a knot portion of a suture or a rope.

32. A method of removing a soft tissue from a patient, the method comprising:
providing a cutting tool having a pair of handles and first and second cutting blades, the first and second cutting blades facing each other;
actuating the pair of handles to bring together the first and second cutting blades to cut away the soft tissue, wherein
the soft tissue is held between the first and second cutting blades in a capture space for removal from the patient.

33. The method of claim 32, wherein the cutting tool is placed to allow in-line cutting of the soft tissue.

34. The method of claim 32, wherein the cutting tool is placed to allow non-in-line cutting of the soft tissue.
